(19) United States
(12) Patent Application Publication
(10) Pub. No.: US 2007/0107723 A1
(43) Pub. Date: May 17, 2007

(54) Pediatric tracheostomy mask

(76) Inventor: Maraha Berg, Orange Park, FL (US)

Correspondence Address: ROGERS TOWERS, P.A., 1301 RIVERPLACE BOULEVARD, SUITE 1500, JACKSONVILLE, FL, 32207, US

(21) Appl. No.: 11/274,390

(22) Filed: Nov. 15, 2005

(51) Int. Cl. A61M 16/00; A62B 7/00

(52) U.S. Cl. 128/200.26; 128/200.24

(57) ABSTRACT

A pediatric tracheostomy mask having a soft main body, a rotating connector member and a rotating tubular adaptor member, wherein the top edge of the main body has a forward extending recess and the upper surface of the main body has a depression extending forward from the recess, and wherein the connector member is provided with a plurality of elongated exhaust openings.

### BACKGROUND OF THE INVENTION

This invention relates generally to the field of medical respiratory devices encompassing devices that create an enclosed area about breathing orifices for the delivery of gases, vapors, treatment mists or the like to the breathing orifices without the need for insertion of tubes into the orifices, and more particularly to the field of such devices that are used in conjunction with tracheostomies.

Patients with respiratory obstructions that interfere with normal breathing are often treated with tracheostomy surgery to provide an opening or stoma through the neck and into the trachea whereby a tracheostomy tube can be inserted to create an alternative breathing passageway. The tube also allows for mechanical ventilation, removal of tracheobronchial secretions, access of instrumentation, delivery of medicine and other procedures. Because the body has no means to naturally moisten the air being delivered through the tube, it is desirable to provide a means to moisten the air prior to it being breathed by the patient.

The common solution to this problem is by the use of a tracheostomy mask that is positioned over the tracheal opening, the mask being held in place by an elastic strap that encircles the patient's neck. The mask is connected to a source of moist air, such as a nebulizer. Typically, the mask comprises a soft, flexible main body with a relatively rigid tubular adaptor joined to the soft body in a manner that allows the tubular adaptor to swivel or rotate such the angle of the tubular adaptor and the connected tube from nebulizer can be altered relative to the patient. An exhaust opening is provided such that exhaled air may exit from the mask. Examples of tracheostomy masks are seen in U.S. Pat. No. 3,236,236 to Hudson, U.S. Pat. No. 3,824,999 to King, U.S. Pat. No. 5,485,837 to Solesee et al., U.S. Pat. No. 5,749,360 to Lacey et al., and U.S. Pat. No. 6,698,426 to Wright.

Because the tracheostomy mask is positioned on the neck of the patient directly beneath the chin, a problem can arise in that the exhaust opening may be inadvertently obstructed by the patient's chin. This is a particular problem in relation to pediatric tracheostomy masks, where the mask is by necessity relatively small in dimension. This potential obstruction problem is especially exacerbated with infants due to the fact that the neck muscles and the ability to control the neck muscles may not be sufficiently developed. To address this, the infant's neck may be secured in a hyper-extended position or large cage-like cover guards may be used. Other problems encountered with traditional tracheostomy masks used in pediatric applications is that the masks can produce pressure sores.

It is an object of this invention to provide a tracheostomy mask for pediatric use that solves the problems set froth above, in that the structural design of the mask minimizes or eliminates the potential for obstruction of the exhaust opening, minimizes or eliminates pressure sores, and provides an improved position for the tubular adaptor relative to the main body of the mask such that a greater range of rotation of the tubular adaptor is present. These objects and other objects not expressed at this time will be readily apparent from the disclosure to follow.

### SUMMARY OF THE INVENTION

A pediatric tracheostomy mask for use with infants and small children, the mask comprising a soft and flexible main body forming a three-dimensional, cup-like area that defines an enclosed area of sufficient size to encompass a tracheostomy opening, a soft, flexible flange member for contacting the patient's skin, a pair of lateral wing members, and a strap attached to the wing members for securing the mask to a patient. The mask further comprises a generally hemispherical, relatively rigid, rotating connector member for connecting a tubular adapter member to the mask, the connector member being mounted to the main body such that the connector member can be rotated about its central axis. The connector member is provided with multiple exhaust apertures in the form of slits. The tubular adaptor comprises an extended linear tube portion with an annular lip at its free end, an elbow portion and a short linear tube portion with a mounting flange for connection to the connector member. The tubular adaptor is rotatably mounted to the connector member.

The flange member is provided with a recess along the upper surface of the main body such that the flange member curves toward the front of the mask. A depression is provided in the upper surface of the main body extending forward from the flange recession. The lowermost point of the junction between the tubular adaptor and the connector member is elevated in comparison to typical masks, with the point of connection being at a point from the bottom of the main body approximately one quarter or more of the overall height of the main body. The plane of rotation of the tubular adaptor is approximately 45 degrees relative to the plane of rotation of the connector member. Preferably, the mask is provided in the shape of an elephant or other animal so as to reduce anxiety of both the patient and the parents.

### BRIEF DESCRIPTION OF THE DRAWINGS

FIG. 1 is a perspective view of a preferred embodiment of the tracheostomy mask of the invention.

FIG. 2 is a front view.

FIG. 3 is a top view.

FIG. 4 is a side view.

FIG. 5 is a cross-sectional view taken along line V-V of FIG. 2, with a portion of the recess and depression features shown for clarity.

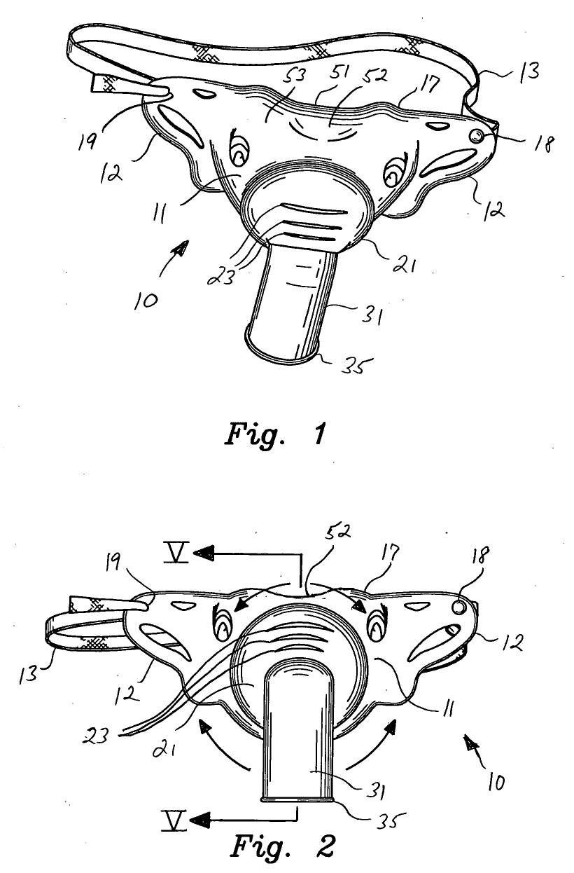

### DETAILED DESCRIPTION OF THE INVENTION

With reference to the drawings, the invention will now be described in detail with regard for the best mode and the preferred embodiment. In general, the invention is an improved pediatric tracheostomy mask, that is, a mask that creates an enclosed environment about the exit port of a tracheostomy tube such that moistened air can be provided for breathing, and one which is down-sized so as to be appropriate for use with infants and small children. The improved pediatric tracheostomy mask provides a better fit to the infant or small child, reduces the likelihood that the exhaust openings will be obstructed by the patient, and provides a more optimum positioning of the tubular adaptor member that connects to the outlet hose of a nebulizer or similar equipment.

For purposes of this disclosure, directions shall be referenced from the orientation of the mask 10 as being attached to a patient sitting, standing or being held upright in a generally vertical manner, such that the front of the mask 10 extends away from the patient and the rear of the mask is in contact with the patient's neck. Likewise, the top of the mask shall mean the uppermost surface facing the chin of the patient and the bottom of the mask shall mean the lowermost surface facing in the direction of the patient's torso. In particular in this disclosure, true vertical shall be taken to mean the plane of rotation of the connector member 21 relative to the main body 11 of the mask 10.

As shown in the figures, the invention is a pediatric tracheostomy mask 10 comprising in general a main body 11, lateral wing members 12, a strap member 13, a rotating connector member 21 and a rotating tubular adaptor member 31. The main body 10 is composed of a soft, flexible plastic and is formed in a three-dimensional, generally cup-like configuration such that the rear of the main body 11 presents a large open area 14. The open rear area 14 is surrounded by a soft, flexible flange member 15 which serves as the contact member between the patient's skin and the mask 10, the flange member 15 and main body 11 being able to conform to the surface shape of the patient's neck due to the material of construction. With the mask in position on the patient's neck, the tracheostomy opening or tube is encircled by the flange member 15 and the main body 11 defines an enclosed space from which moistened air or other chosen gas or vapor material is drawn into the tracheostomy tube and through which exhaust air can be exhaled. The main body 11 has a bottom edge 16 and a top edge 17 comprising the lower and upper portions of the flange member 15.

Extending from each side of the main body 11 are lateral wing members 12, preferably composed of a soft, flexible material similar or identical to the material composing the main body 11. The wing members 12 are generally triangularly shaped and a strap connecting means is provided in each wing member 12 for connecting a strap member 13, such that the strap member 13, preferably composed of an elastic band, foam, fabric or the like, can be positioned bout the patient's neck to temporarily secure the mask 10. While the strap member 13 may be permanently affixed to the wing members 12, preferably strap member 13 is removably attached such that its length can be adjusted and such that at least one, and preferably, both ends of the strap member 13 can be removed from the wing members 12. As shown in the drawings, the strap connecting means may comprise a mechanical fastener 18 and a strap aperture 19. With this structure, the strap member 13 can be drawn through the strap aperture 19 and tied or knotted once the proper length is achieved.

Connector member connecting means are provided such that a connector member 21 may be rotatably mounted onto the front of the main body 11. The connector member connecting means as shown comprises in combination an annular internal flange member 41 disposed about a circular opening 40 in the front of the main body 11 and an annular channel member 42 disposed about a circular opening in the rear of the connector member 21 and adapted to receive the annular internal flange member 41, but it is to be understood that other structures may be provided for the connector member connecting means. Connector member 21 is preferably composed of a plastic material more rigid than that of the main body, the material having sufficient rigidity such that little or no deformation occurs under finger pressure. Connector member 21 is preferably circular in configuration at its rear, such that the junction of the connector member 21 with the main body 11 defines a substantially vertical plane of rotation, the interconnection of the annular interior flange member 41 and the annular channel member 42 enabling the connector member 21 to be rotated relative to the main body 11, preferably over a range of 360 degrees. The spherical forward portion of the connecting member 21 is provided with a circular opening 22 defined by tubular adaptor connecting means. Above the circular opening 22 are positioned a plurality of elongated, generally horizontally disposed exhaust openings 23, preferably in the configuration of slits of decreasing length from the uppermost slit to the lowermost slit. The exhaust openings 23 provide means for the discharge of exhaled air, the elongated configuration being less likely to be obstructed or occluded, such as could occur with standard circular exhaust openings should an infant insert a finger into the circular opening.

A tubular adaptor member 31, being the direct means for connecting a nebulizer tube or the like to the mask 10 and composed of a relatively rigid material similar or identical in physical properties to the material of composition of the connector member 21, is rotatably mounted to the connector member 21 by tubular adaptor connecting means, which as shown comprises in combination an annular rim member 43 which defines the circular opening 22 and an annular channel member 44 that receives the annular rim member 43. As before, it is to be understood that other structures may be provided for the tubular adaptor connecting means. The tubular adaptor member 31 comprises an elongated, preferably linear, tube portion 32, and a relatively short, preferably linear, tube portion 34 connected together at an elbow or curved joint 33, whereby the central axis of the elongated linear portion 32 is not parallel to the central axis of the short tube portion 34. Preferably, the angle formed between the elongated tube portion 32 and the short tube portion 34 is approximately 45 degrees. The tubular adaptor member 31 is mounted to the connector member 21 such that the tubular adaptor member 31 may rotate relative to the connector member 21 about the central axis of the short tube portion 34, preferably over a range of 360 degrees. The plane of rotation defined by the junction 54 of the tubular adaptor connector means is preferably at approximately 45 degrees to the plane of rotation defined by the junction 55 of the connector member connecting means, and therefore the plane of rotation defined by the tubular adaptor connector means is also approximately 45 degrees from vertical. In this manner the position of the tubular adaptor member 31, by rotation of the connector member 21 and or rotation of the tubular adaptor member 31, can be altered in infinite ways so as to provide the most comfortable and least obstructed path and position for the nebulizer tube. The tubular adaptor member 31 is provided with an annular lip member 35 on its free end to better secure the nebulizer tube.

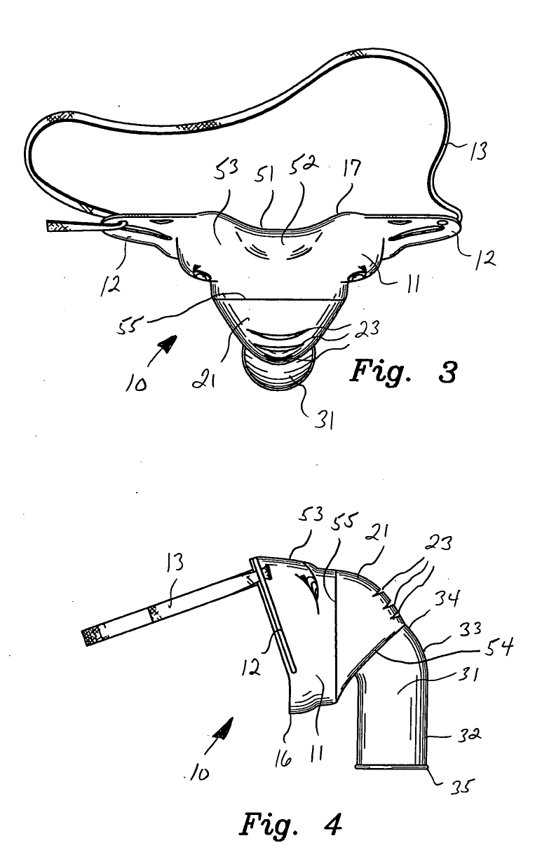

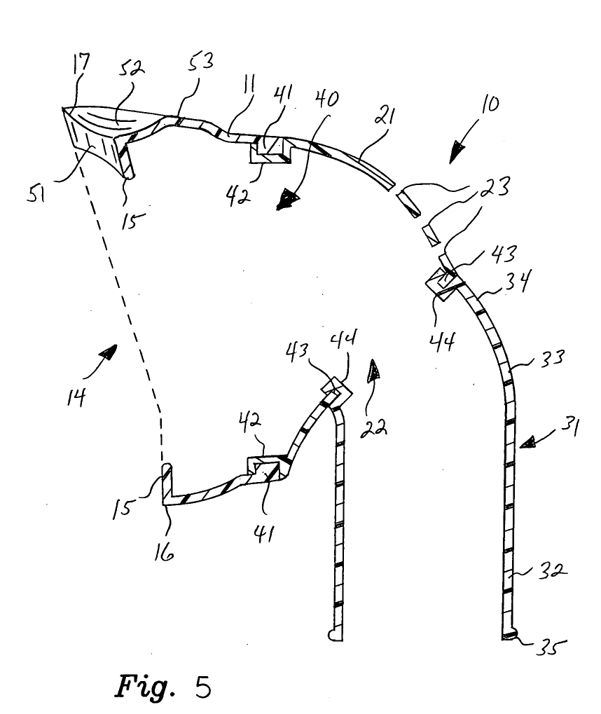

To address the problems of known pediatric tracheostomy masks, it is imperative that the mask 10 be structured to better fit the confines of an infant or small child neck and chin area. To this end, the central portion of the top edge 17 of the main body 11 and the corresponding central portion of the flexible flange member 15 are provided with a forward extending recess 51, such that a pronounced concave configuration is imparted in the top edge 17 when viewed from above, as shown best in FIG. 3. Additionally, a concave depression 52 is provided in the upper surface 53 of the main body 11, the depression 52 extending forward from the recess 51. The combination of the recess 51 and depression 52 account for the proximity of the chin of the infant patient to the mask 10, given the relatively short neck length. This short neck length is also addressed by dimensioning the mask 10 such that the distance between the bottom edge 16 of the main body 11 and the lowermost point of the junction 54 between the tubular adaptor member 31 and the connector member 21, with the connector member 21 rotated relative to the main body 11 such that the tubular adaptor member 31 is at its lowermost position, is approximately one quarter or more the overall height of the main body 11. With this structure, the junction between the tubular adaptor member 31 and the connector member 21 is located at a higher position on the main body 11 than found on standard masks.

As a representative example, suitable approximate dimensions for the mask 11 consist of a height of 1.893 inches for the main body 11, a tubular adaptor member 31 having an elongated tube portion of 1.13 inches and a diameter of 0.1875 inches, where lowermost point of the junction between the tubular connector 31 and the connector member 21 is 0.459 inches above the bottom edge 16 of the main body 11.

Preferably, the mask 10 is provided with features that provide the impression of an animal character. As shown, the mask 10 can be adapted to give the appearance of an elephant head by configuring the lateral wing members 12 in the shape of elephant ears and providing markings that simulate eyes. The purpose of this adaptation is to present a less threatening image for the mask 10, which can be soothing for both the patient and the patient' parents and siblings.

It is understood that equivalents and substitutions for certain elements and components set forth above may be obvious to those skilled in the art, and therefore the true scope and definition of the invention is to be as set forth in the following claims.

I claim:

1. A pediatric tracheostomy mask comprising:
a main body composed of a soft, flexible plastic, said main body comprising an open rear area, a peripheral flange member, a bottom edge, a top edge and an upper surface;
lateral wings members joined to said main body;
a strap member connected to said lateral wing members;
a connector member rotatably mounted on said main body;
a tubular adaptor member rotatably mounted on said connector member;
said main body further comprising a forward extending recess disposed in said top edge and a depression disposed on said upper surface and extending forward from said recess.

2. The mask of claim 1, wherein said lateral wing members are composed of a soft, flexible plastic.

3. The mask of claim 1, wherein said strap member is an elastic band.

4. The mask of claim 1, wherein said strap member is removably connected to said lateral wing members.

5. The mask of claim 1, wherein said connector member and said tubular adaptor member are composed of rigid plastic.

6. The mask of claim 1, wherein said mask has the appearance of an elephant head.

7. The mask of claim 1, wherein said tubular adaptor member comprises an elongated linear tube portion, an elbow joint and a short linear tube section.

8. The mask of claim 1, wherein said tubular adaptor member is connected to said connector member at a junction defining a plane of rotation, and wherein said connector member is connected to said main body at a junction defining a plane of rotation, and further wherein said plane of rotation of said junction between said tubular adaptor member and said connector member is at approximately 45 degrees to said plane of rotation of said junction between said connector member and said main body.

9. The mask of claim 1, said connector member further comprising a plurality of elongated exhaust openings.

10. The mask of claim 9, wherein said lateral wing members are composed of a soft, flexible plastic.

11. The mask of claim 9, wherein said strap member is an elastic band.

12. The mask of claim 9, wherein said strap member is removably connected to said lateral wing members.

13. The mask of claim 9, wherein said connector member and said tubular adaptor member are composed of rigid plastic.

14. The mask of claim 9, wherein said mask has the appearance of an elephant head.

15. The mask of claim 9, wherein said tubular adaptor member comprises an elongated linear tube portion, an elbow joint and a short linear tube section.

16. The mask of claim 9, wherein said tubular adaptor member is connected to said connector member at a junction defining a plane of rotation, and wherein said connector member is connected to said main body at a junction defining a plane of rotation, and further wherein said plane of rotation of said junction between said tubular adaptor member and said connector member is at approximately 45 degrees to said plane of rotation of said junction between said connector member and said main body.

17. The mask of claim 1, wherein said tubular adaptor member is connected to said connector member at a junction, wherein the distance between said bottom edge of said main body and the lowermost point of said junction between said tubular connector and said connector member, with said connector member rotated relative to said main body such that the tubular adaptor member is at its lowermost position, is approximately at least approximately one quarter the overall height of said main body.

18. The mask of claim 9, wherein said tubular adaptor member is connected to said connector member at a junction, wherein the distance between said bottom edge of said main body and the lowermost point of said junction between said tubular connector and said connector member, with said connector member rotated relative to said main body such that the tubular adaptor member is at its lowermost position, is approximately at least approximately one quarter the overall height of said main body.
